(19) United States
(12) Patent Application Publication
(10) Pub. No.: US 2021/0205087 A1
(43) Pub. Date: Jul. 8, 2021

(54) JOINT IMPLANT FOR NEW TISSUE FORMATION AT THE JOINT

(71) Applicant: Christoph KARL, Kreuzlingen (DE)

(72) Inventors: Christoph KARL, Kreuzlingen (CH); Vilma METHNER, Kleinmachnow (DE)

(73) Assignee: Christoph KARL, Kreuzlingen (DE)

(21) Appl. No.: 17/059,689

(22) PCT Filed: Apr. 30, 2019
(86) PCT No.: PCT/EP2019/061066

§ 371 Date: Nov. 30, 2020

(30) Foreign Application Priority Data
Jun. 11, 2018 (DE) 10 2018 113 809.1

(51) Int. Cl. A61F 2/30; A61L 27/34; A61L 27/22; A61L 27/18; A61L 27/06; A61L 27/04; A61L 27/10; A61L 27/02; B33Y 80/00

(52) U.S. Cl. CPC A61F 2/30; A61L 2300/414; A61L 27/34; A61L 27/227; A61L 27/18; A61L 27/06; A61L 27/045; A61L 27/10; A61L 27/025; B33Y 80/00; A61F 2002/30985; A61F 2002/30766; A61F 2002/3084; A61F 2002/30205; A61F 2002/30224; A61F 2/30756

(57) ABSTRACT

The invention relates to a joint implant for new tissue formation at a joint, wherein the joint implant (1) comprises a rod-shaped body with a base area (11), a cover area (12) and a sleeve area (13), wherein at least the cover area (12), in particular the entire rod-shaped body, of the joint implant (1) has a hydrophobic surface for facilitating chondrocyte differentiation of mesenchymal stem cells, and a thread structure (15) is at least partially formed on the sleeve area (13) of the joint implant (1).

The present invention relates to a joint implant for new tissue formation at the joint, in particular on a joint implant for new cartilage formation at the knee, hip, shoulder, ankle, metatarsophalangeal or hand joint.

Joint diseases (arthrosis) are among the ten most common disease types worldwide. Arthrosis is painful, and without treatment, it can lead to immobility of the affected joints and even to total joint replacement. Total replacement of the affected joint is quite costly and ordinarily places a psychological burden on the patient. Revisions of the joint replacement involve further costs, stress for the patient, and often complications. Various approaches have therefore been developed for the curative treatment of joint diseases (arthrosis) and in particular for the curative treatment of knee and hip joint arthrosis in order to avoid joint replacement.

Mammals cannot readily repair cartilage and bone defects or losses alone. However, the tissue deficit can be filled by specialised cells or stem cells that develop into such specialized cells. Furthermore, cartilage has no access to the blood and bone marrow. It is supplied via diffusion from the surrounding tissue. However, when this surrounding tissue itself is damaged, access to tissue and cells capable of regeneration is insufficient.

The approaches currently available for the curative treatment of joint diseases are explained below.

Drug treatment options: Drug treatment options are limited to the symptomatic use of anti-inflammatories and analgesics and sometimes intra-articular, sometimes systemic treatment with hyaluronic acid, chondroitin sulphate, interleukin-1 receptor antagonists and glucosamine sulphate. Although such treatment has shown good results in pain reduction, it has not yet been successful in preventing the progression of arthrosis.

Surgical therapies: A distinction is made between joint-preserving and joint-replacement surgery. Joint replacement with partial and full prostheses of metal and ceramics results in loss of the joint due to use of such prostheses. Prostheses are anchored to the bone, and depending on the age of the patient, must be replaced every 10 to 15 years. During such replacement, bone shafts are often deepened and further tissue is removed.

The joint-preserving operations described below offer a wide variety of possibilities for regeneration.

Surgical therapies such as local bone or cartilage transplantation or autologous chondrocyte transplantation or implantation (ACT or ACI are used as synonyms) have not yet become established, because such therapies require two operations (removal and re-implantation), which means that the joint bears no load or is immobilized during rehabilitation. Moreover, the still-healthy cartilage at the removal site is also damaged.

The most widespread therapies are therefore surgical therapies such as so-called Pridie drilling, anterograde/retrograde drilling and microfracturing. In these surgical therapies, the local cartilage is not replaced; rather, for example, multiple perforations are carried out through the subchondral border lamella. In Pridie drilling and microfracturing, which is a further development thereof, bleeding into the cartilage defect can be achieved in the defect area, allowing mesenchymal stem cells from the cancellous space, fibroblasts and chondrocytes to wash into the cartilage defect. These combine with growth factors to form a blood clot (“super clot”) and differentiate into articular cartilage. Clinical studies have shown reduction of pain and favourable joint mobility. However, the long-term removal of weight or immobilization of the joint is a problem in this case as well, leading as a rule to the development of poor-quality fibrous cartilage. Due to its structure, this cartilage is often insufficient to support the high mechanical loads placed on the knee joint in particular and degenerates rapidly, which can necessitate further surgical interventions.

For this reason, carbon rods have been developed as joint implants for new tissue formation at the joint that are placed in the bore holes and are designed to be rapidly overgrown.

EP 1450875 A1 discloses such a joint implant for new tissue formation at the joint, wherein the rods used are composed of densified carbon with a predetermined porosity. The use of such conventional carbon rods also allows fibroblasts and mesenchymal stem cells to be washed from the cancellous space into the cartilage defect, forming a “super clot” and differentiating into articular cartilage.

Because of two significant drawbacks, however, this system has not yet become established. On the one hand, carbon is not accepted by orthopaedic surgeons as an active component for use in cartilage because of the risk of microabrasion. On the other hand, the surface is not designed for colonisation by stem cells, which also manifests itself in the development of poor-quality regenerative fibrous cartilage.

Because of these limitations, none of the above-mentioned therapies have yet become established as a standard of care.

The object of the invention is therefore to provide a joint implant for new tissue formation at the joint that provides improved properties in new tissue formation and handling.

According to the invention, this object is achieved by means of the features of claim 1.

In particular, because a rod-shaped body is used in which at least the cover area of the joint implant comprises a hydrophobic surface for purposes of chondrocyte differentiation and thus facilitates the chondrogenesis of mesenchymal stem cells and a thread structure is at least partially formed on the sleeve area of the joint implant, one can achieve an improvement in new tissue formation and in particular new cartilage formation at the joint and the formation of higher-quality weight-bearing cartilage. The use of the implant according to the invention allows cartilage regeneration to be achieved that is long-lasting, and because of the continuous transport of mesenchymal stem cells in the direction of the joint space, sustainable, and this cartilage regeneration can either allow further surgery on the joint to be delayed or obviate the need for such surgery. In particular, the thread structure facilitates the placement and removal of the rod-shaped body, and the enlarged surface of the rod-shaped body allows improved transport of cells from the bone tissue to the tissue surface (cartilage). In addition, with the joint implant according to the invention, one can achieve reliable fixation of so-called scaffolds (carrier materials) and tissues such as tendons and ligaments to the bone.

Preferably, the rod-shaped body, and in particular the cover area and the thread structure, are produced via an artificial trabecular structure by means of a 3D printing process, which allows the structure and dimensioning of the rod-shaped body to be defined in a highly precise manner and further improves the transport or lifting function for the mesenchymal stem cells.

Preferably, the rod-shaped body is a hollow body, by means of which the lifting function for mesenchymal stem cells is further improved.

For example, the material of the rod-shaped body can comprise a non-bioresorbable polymer, in particular PA, PEK, PEKK, PEEK or UHMWPE, or a bioresorbable polymer, e.g. PCL, a metal, in particular Ti, in particular pure titanium grade 1, stainless steel, or a metal alloy, in particular Ti6Al4V (also referred to as Ti64), in particular CoCr, or a non-bioresorbable ceramic, in particular Al₂O₃ or ZrO₂, or a bioresorbable ceramic, in particular Ca₃(PO₄)₂, or a combination of these materials, thus making it possible to implement a high-mechanical-strength joint implant with improved properties for new tissue formation. Preferably, the material of the rod-shaped body can comprise Si₃N₄.

Preferably, the hydrophobic surface can be configured as a hydrophobic chemical coating on a hydrophilic porous substrate, thus allowing chondrocyte differentiation to be implemented in a particularly simple and effective manner.

For example, at least the cover area of the joint implant can comprise a recess for applying torque, thus making it possible to carry out particularly precise placement in the bone.

Preferably, the recess has the form of a slot or a cross slot, thus obviating the need for special tools for the placement or removal of the implant. Alternatively, the recess can also have the form of an inner polygon or Torx.

Preferably, the thread structure of the joint implant can have a thread pitch of 0.5 mm to 1 mm, thus making it possible to achieve particularly favourable transport properties for mesenchymal stem cells together with outstanding mechanical properties.

For example, the base area of the joint implant can have a point, and the thread structure can have a self-forming, self-tapping or self-cutting configuration, thus further simplifying placement of the implant and in particular obviating the need for drilling the bone.

For example, the hydrophobic surface can be formed by a hydrophobic chemical coating and in particular a segmented polymer, preferably a polyurethane or polyelectrolyte or a hydrophobically functionalized chitosan or chitosan derivative.

In this manner, the lifting function for the transport of mesenchymal stem cells is further improved.

Alternatively or additionally, the hydrophobic surface can be implemented by hydrophobic nanostructuring of a substrate, which further facilitates chondrocyte differentiation and the transport of stem cells.

Preferably, the rod-shaped body, in particular in the cover area, can further comprise a coating with a growth factor, thus further improving the differentiation of cartilage and the growth of tissue, and in particular of cartilage material on the joint implant and at the defect site.

Moreover, the rod-shaped body, in particular in its base area, can comprise a hydrophilic surface for facilitating osteoblast differentiation of mesenchymal stem cells, thus improving bone formation and thus anchoring in the bone.

Further advantageous embodiments of the invention are characterized in the further dependent claims.

The invention is described in further detail below using examples with reference to the drawing.

The figures show the following:

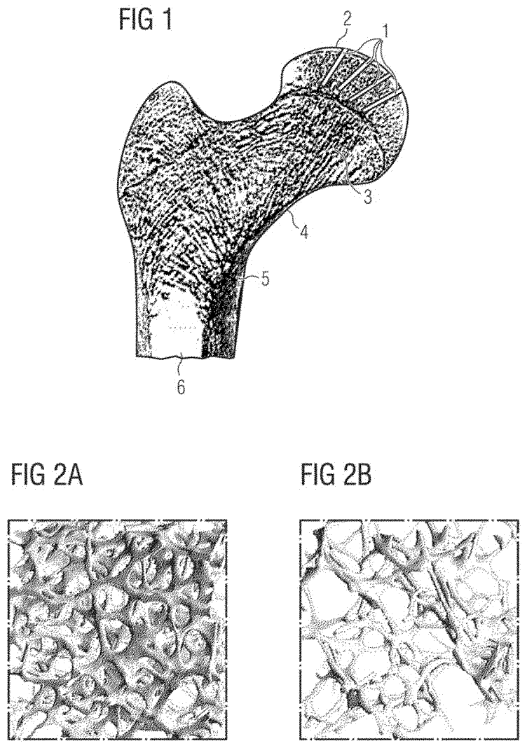

FIG. 1 is a simplified sectional view of a femoral bone with joint implants according to the invention;

FIGS. 2A and 2B are simplified perspective views of natural trabecular structures;

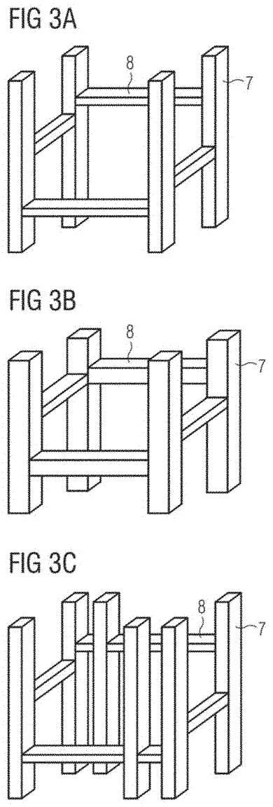

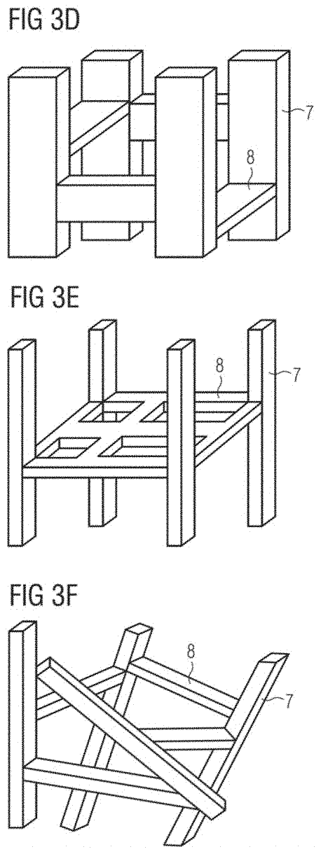

FIGS. 3A to 3F are simplified perspective views of artificial trabecular structures according to examples of the invention;

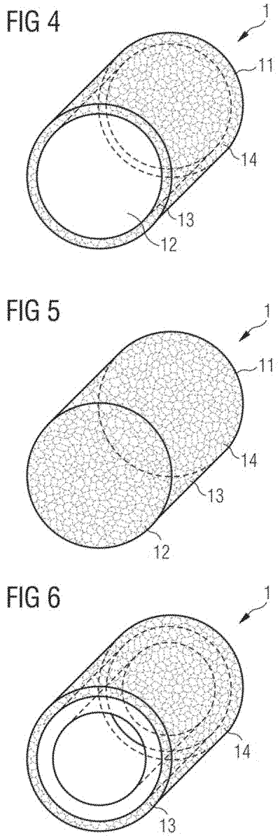

FIG. 4 is a simplified perspective view of a base structure of a joint implant according to a first example of the invention;

FIG. 5 is a simplified perspective view of a base structure of a joint implant according to a further example of the invention;

FIG. 6 is a simplified perspective view of a base structure of a joint implant according to a further example of the invention;

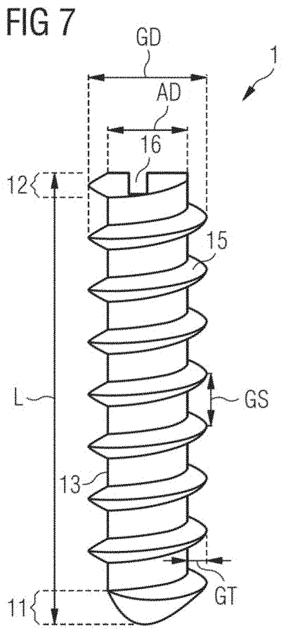

FIG. 7 is a simplified side view of a joint implant according to a preferred example of the invention;

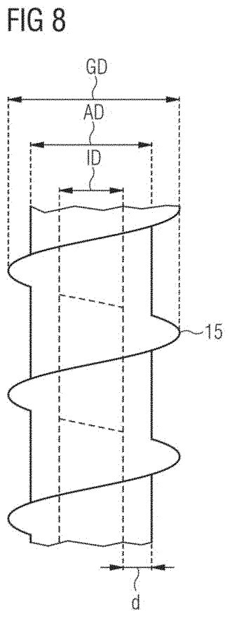

FIG. 8 is a simplified partial view of the joint implant according to FIG. 7;

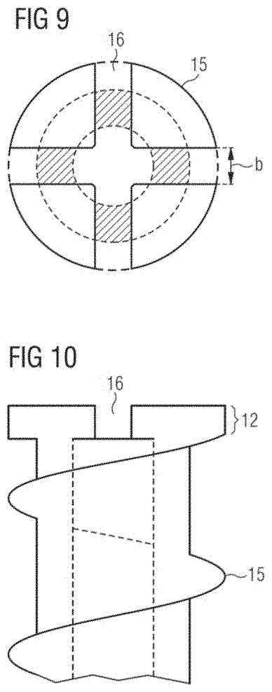

FIG. 9 is a simplified top view of the joint implant according to FIG. 7;

FIG. 10 is a simplified partial side view of the cover area of the joint implant according to FIG. 7;

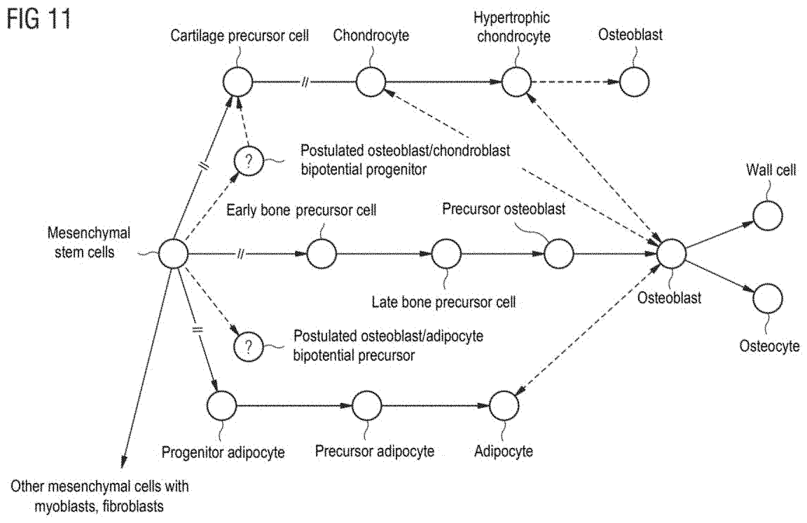

FIG. 11 is a schematic overview of the various differentiation stages of mesenchymal stem cells according to Aubin 1998;

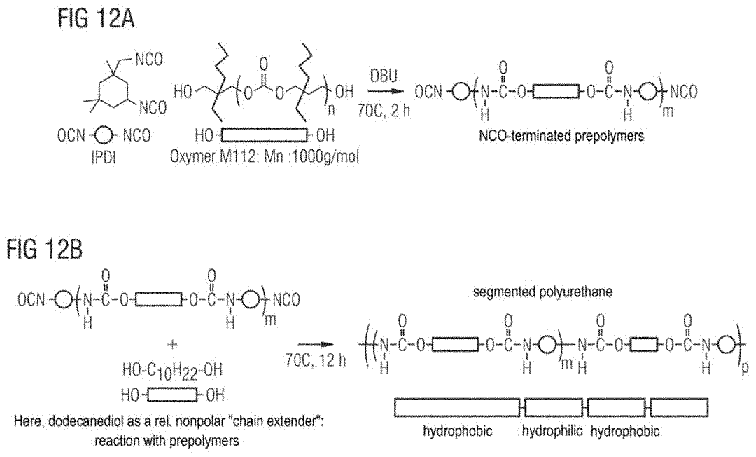

FIGS. 12A and 12B illustrate the production of a hydrophobic chemical coating using the example of segmented polyurethanes.

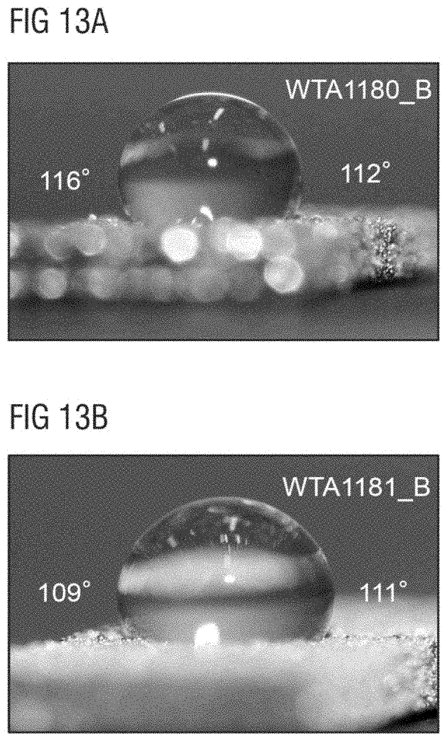

FIG. 13A and 13B are enlarged views of Ti substrates coated with segmented PU in order to illustrate respective contact angles.

FIG. 1 shows a simplified sectional view of an upper section of a femoral bone as an example illustrating the use of the joint implants according to the invention for new tissue formation at a femoral/hip joint. In FIG. 1, reference number 1 denotes the joint implants according to the invention, which can be inserted in the area of the joint into the femoral bone. In this process, for example in damaged cartilage areas 2, one or a plurality of indentations can be drilled, stamped, or otherwise formed in the bone, after which a respective joint implant 1 can be inserted in the respectively formed indentation. The respective indentation is preferably dimensioned such that the joint implant 1 used or the cover area thereof does not protrude at the surface of the bone or cartilage 2, but is flush therewith, or the cover area of the joint implant 1 is preferably below the surface of the bone or cartilage 2. Alternatively, the joint implant according to the invention can also be screwed without forming an indentation directly into the damaged area. The articular cartilage 2 damaged because of joint disease (arthrosis) can be at least partially regenerated by means of the joint implants according to the invention 1, as new formation of tissue, and in particular of articular cartilage, takes place at the ends or in the cover areas of the inserted joint implants 1.

According to FIG. 1, the bone comprises a periosteum 4 covering the bone, wherein a natural trabecular structure is present at the end areas 3 of the bone that is referred to as the so-called spongiosa. Furthermore, in its middle area, the bone comprises relatively solid cortical bone 5 and in its interior a medullary cavity 6.

By using an artificial trabecular structure at least on one surface of the joint implant 1, improved properties of new tissue formation at the joint can be achieved, which will be explained in detail below.

FIGS. 2A and 2B show simplified perspective views of natural trabecular structures, such as those present for example in the cancellous bone area 3 of the human femoral bone. As shown in FIG. 2A, the cancellous bone area 3 of a young, healthy person is permeated with a highly fine and dense natural trabecular structure, while as shown in FIG. 2B, an older person, in particular suffering e.g. from osteoporosis, often shows a sharply altered natural trabecular structure with only a few, very thin trabeculae in the cancellous bone area 3.

For example, the joint implant 1 can comprise a rod-shaped body that has an artificial trabecular structure at least on its surface or also overall. The artificial trabecular structure, which is at least partly open or permeable to fluids, of the joint implant 1 allows for example rapid colonisation of the trabecular surface and in particular the cover area of the rod-shaped body with cartilage-forming cells such as e.g. chondrocytes, which results in significantly accelerated and at the same time long-lasting overgrowth and further allows the formation of high-quality regenerative cartilage.

Preferably, the joint implant 1 with its artificial trabecular structure can be constructed and macro- and microstructured with respect to the biomedical application by means of suitable 3D printing processes such that a specified internal and external structure and roughness results in optimum anchoring to the natural trabecular structure in the indentation (drilling, stamping etc.) in the cancellous bone area 3. In this manner, vertical, lateral, and optionally rotational mobility of the joint implant 1 in the indentation can be ruled out.

FIGS. 3A to 3F show simplified perspective views of artificial trabecular structures according to examples of the present invention. The artificial trabecular structure comprises a plurality of rod-shaped or plate-shaped elements (trabeculae), which when connected to one another give rise to a 3-dimensional microarchitecture.

The artificial trabeculae, which are preferably produced by 3D printing technology and biomimetic, may not show values below or above certain parameters.

In the following, the essential parameters of the artificial trabecular structure according to the invention are defined in further detail.

The so-called mean trabecular thickness (Tb.Th) defines the average trabecular thickness of the respective trabeculae or rod-shaped elements. For example, as the respective trabeculae according to FIG. 3A can have different forms, Tb.Th constitutes the average of the local thicknesses of all of the artificial trabeculae. The local thickness is derived e.g. in rectangular trabeculae from the trabecular diagonal and in circular trabeculae from the trabecular diameter. FIG. 3B shows a schematic diagram of the effects on the artificial microstructure of an increase in the average trabecular thickness Tb.Th. Preferably, the average trabecular thickness

Tb.Th for the artificial trabecular structure is in the range of 100 to 500 μm, and in particular 150 to 400 μm.

The so-called mean trabecular separation (Tb.Sp) defines the average trabecular separation analogously to the average trabecular thickness Tb.Th. A decrease in Tb.Sp can result from a change in various other parameters, e.g. an increase in Tb.Th (FIG. 3B), a decrease in Tb.N (FIG. 3C) or an increase in the structure model index (SMI) (FIG. 3D). The unit of average trabecular separation Tb.Sp is the pm and for the artificial trabecular structure of the present invention is preferably in a range of 100 μm to 900 μm, and in particular 200 μm to 600 μm.

The so-called trabecular number (Tb.N) is defined as the inverse function of the average distance between the axes of the plates and/or rods and indicates the number of trabeculae per mm. FIG. 3C shows for example an increase in Tb.N compared to FIG. 3A. Preferably, the trabecular number Tb.N of the artificial trabecular structure is in a range of 1 to 6 per mm, in particular in a range of 1.6 to 5.2 per mm.

The so-called “structure model index” (SMI) is a further descriptive parameter of the artificial trabecular structure, which for example can be a network composed of plate-like and rod-like elements. In fact, however, the trabecular network is not of one form or the other; rather, there is a fluid transition between them. With increasing age, for example, a more plate-like network is converted to a more rod-like one. Based on this realization, the so called structure model index (SMI) was introduced, which makes it possible to quantify the structure with respect to the number of plates and rods. For an ideal plate model, the SMI has a value of 0 (i.e. a pure plate structure), and for an ideal rod model, the value is 3. The SMI thus describes the relative composition of the artificial trabecular structure of plates and rods. FIG. 3D schematically shows a decrease in SMI. The SMI is dimensionless and for the present invention is for example 0.2 to 2.0, and preferably 0.25 to 1.8.

So-called connectivity-density (Conn.D) is a measure of the connectivity of the trabecular network. Connectivity is the maximum number of connections that can be disrupted within the network, e.g. by microfractures, without separating the network as a whole into two parts that are no longer connected to each other. FIG. 3E is a schematic diagram of an increase in connectivity-density Conn.D. Preferably, the connectivity density Conn.D of the artificial trabecular structure of the present invention is in a range of 1/mm³ to 60/mm³, in particular 1.5/mm³ to 45/mm³.

The geometric degree of anisotropy (DA) is a parameter for quantifying spatial asymmetry. The higher the DA, the greater the orientation of the artificial trabecular structure in a specified direction. FIG. 3F shows a schematic diagram of a decrease in DA. Like the parameter SMI, DA is also dimensionless. A DA of 0 indicates a perfectly isotropic structure, and a DA of 1 a perfectly anisotropic structure. Additionally, the degree of anisotropy is also indicated by the so-called tDA (alternative DA) with values ranging from 1 for perfectly isotropic to infinity for perfectly anisotropic. However, the tDA is not used here in describing the structure according to the invention. Preferably, the geometric degree of anisotropy DA for the artificial trabecular structure of the present invention is in a range of 0.1 to 1.0, in particular 0.2 to 0.8 and more preferably 0.2 to 0.6.

The so-called bone volume/tissue volume fraction (BV/TV) is the unit trabecular volume per total unit volume of a trabecular structure under consideration. An increase in BV/TV can result from a change in various other parameters, e.g. an increase in Tb.Th (FIG. 3B), an increase in Tb.N (FIG. 3C) or a decrease in SMI (FIG. 3D). Preferably, the BV/TV of the trabecular structure according to the invention is in a range of 6% to 70%, and more preferably 20 to 50%.

Finally, the so-called marrow star volume (MSV) defines a respective trabecular porosity of the artificial trabecular structure. More precisely, the MSV determines the size of the hollow spaces in the artificial trabecular structure. The arithmetic mean mMSV according to the invention is preferably in a range of 0.05 mm³ to 110 mm³, in particular 0.05 mm³ to 9 mm³ and more preferably 0.05 mm³ to 5 mm³.

FIG. 4 shows a simplified perspective view of a base structure of the joint implant 1 according to a first example of the invention. The joint implant 1 comprises a rod-shaped body in the form of a solid (fluid-tight) cylinder comprising a base area 11, a sleeve area 13 and a cover area 12. The joint implant 1 can be used in the bone according to FIG. 1 in such a manner that its cover area 12 in the area of the cartilage 2 is preferably arranged slightly recessed in the bone. This allows the cover area 12 of the joint implant 1 to act as a growth area for the tissue or articular cartilage to be newly formed 2. The base area 11 and the lower part of the joint implant are preferably located partially or completely in the cancellous bone area 3.

As shown in FIG. 4, the above-described artificial trabecular structure 14 can be formed at least in a sleeve area 13. Furthermore, however, the cover area 12 in particular can also comprise the artificial trabecular structure 14. For example, a trabecular structure of the joint implant 1 that is open and permeable to bodily fluids allows rapid colonisation of the trabecular surface with cells such as chondrocytes, which results in significantly accelerated overgrowth. Furthermore, depending on its structure and coating, the joint implant according to the invention allows the growth of regenerative fibrous cartilage, or even high-quality hyaline regenerative cartilage, particularly on the cover area 12.

FIG. 5 shows a simplified perspective view of a base structure of the rod-shaped body of the joint implant 1 according to a further example of the invention, wherein the same reference numbers indicate the same or corresponding elements, and a repeated description thereof will therefore be dispensed with below. According to FIG. 4, the rod-shaped body of the joint implant 1 can comprise a solid cylinder, wherein the trabecular structure according to the invention 14 is formed at least on the sleeve surface 13 thereof, whereas according to FIG. 5, the rod-shaped body of the joint implant 1 can also consist completely of the artificial trabecular structure 14. In this manner, the washing in, proliferation, and migration of fibroblasts, mesenchymal stem cells from the cancellous bone area 3 and chondrocytes into the cartilage defect can be further improved.

FIG. 6 shows a simplified perspective view of a base structure of the rod-shaped body of the joint implant 1 according to a further example of the invention, wherein the same reference numbers indicate the same or corresponding elements, and a repeated description thereof will therefore be dispensed with below. According to FIG. 6, the rod-shaped body of the joint implant 1 preferably comprises a hollow body, such as e.g. a hollow cylinder, wherein the artificial trabecular structure 14 is formed at least in a sleeve area 13 of the hollow cylinder. For example, the hollow body comprises at least one continuous hollow space or a plurality of non-continuous hollow spaces. Alternatively, however, the hollow cylinder can also be composed completely of the artificial trabecular structure 14 (not shown). The washing in, proliferation, and migration of fibroblasts, mesenchymal stem cells from the cancellous bone area 3 and chondrocytes into the cartilage defect can be further improved by this enlargement of the interaction surface.

FIG. 7 shows a simplified side view of a joint implant according to a preferred example of the invention, wherein the same reference numbers indicate the same or corresponding elements, and a repeated description thereof will therefore be dispensed with below. According to FIG. 7, a thread structure 15 is at least partially formed on the sleeve area 13 of the rod-shaped body. The thread structure 15 either extends continuously from the base area 11 to the cover area 12 or has interruptions. The thread structure 15 can also be configured only from the base area 11 to approx. the middle of the rod-shaped body on the sleeve area 13 or alternatively only from approx. the middle of the rod-shaped body to the cover area on the sleeve area 13. The rest of the rod-shaped body then remains unthreaded.

The thread structure 15 can for example be formed from the above-described artificial trabecular structure, but it can also be formed from a solid material.

According to FIG. 7, a preferred macrostructuring can be implemented for example by means of a 3D printing process, wherein a thread diameter GD of the thread structure 15 is preferably 3 mm. A thread depth GT of the thread structure 15 is preferably 0.5 mm and a thread pitch GS of the thread structure 15 preferably in the range of 0.5 mm to 1 mm. An external diameter AD of the rod-shaped body is for example 2 mm.

Furthermore, a recess 16 for applying torque can be configured at least in the cover area 12 of the joint implant 1. The recess 16 is configured by means of macrostructuring, again preferably using a 3D printing process. For example, as a recess 16, a slot is formed for receiving a screwdriver or bit that can be used to screw the joint implant into the bone. A special application system is thus unnecessary, as available medical screwdrivers can be used. This substantially reduces costs.

Preferably, the rod-shaped body of the joint implant 1 has a length L of at least 0.6 cm and at most 1.2 cm for application in the patella and extremely small joints such as the hand or ankle joints and at least 0.8 cm and at most 2.2 cm, in particular 1.0 cm to 1.6 cm and more preferably 1.25 cm, for proximal and distal tibial and femoral application respectively in the knee and hip joint. This allows optimal accessibility and growing-in of mesenchymal stem cells. The rod-shaped body of the joint implant 1 can further have a thread diameter GD of at least 2 mm and at most 6 mm, preferably 3 mm, thus making it possible to achieve an optimal lateral surface facing the synovia for the formation of replacement cartilage tissue. The thickness of the artificial trabecular structure 14 (e.g. of the sleeve area 13) is preferably 0.5 to 2.0 mm, and more preferably 0.5 to 1.5 mm.

Moreover, the joint implant 1 according to FIG. 7 can have a point or a cone in the base area 11, and the thread structure 15 can have a self-forming, self-tapping or self-cutting configuration. In this case, the formation of an indentation or drilling into the bone can be dispensed with, as the joint implant 1 automatically digs into the bone.

By means of a defined and repetitive mesh structure of the artificial trabecular structure that mimics the natural trabecular structure, a channel structure with a correspondingly adapted shape of the joint implant 1 or a combination of a mesh structure and a channel structure, one can achieve optimum growth of endogenous tissue into the boundary volume between the joint implant 1 and the indentation or drill channel, in particular into the internal volume of the sleeve area 13 of the joint implant 1 and the end of the joint implant 1 facing the synovia (joint space).

By means of the thread structure 15, the joint implant 1 can be very precisely positioned in the bone and adjusted with respect to its height. Furthermore, the thread structure 15 provides improved mechanical anchoring in the bone. In addition, the joint implant 1 can be removed without having to be milled out over a large area. However, the thread structure 15 enlarges the surface of the joint implant 1 in a particularly advantageous manner, thus further improving the transport (lifting function) of cells. The special shape of the thread structure 15 thus allows even more cells from the bone tissue to reach the tissue surface, which results in further improved cartilage formation and thus regeneration.

FIG. 8 shows a simplified partial view of the joint implant according to FIG. 7, wherein the same reference numbers indicate the same or corresponding elements, and a repeated description thereof will therefore be dispensed with below. According to FIG. 8, a hollow cylinder can preferably be used as a rod-shaped body, which further improves the transport (lifting function) of cells from the bone tissue to the tissue surface. An internal diameter ID of the rod-shaped body is preferably 1 mm, and a thickness d of the sleeve area 13 is preferably 0.5 mm. For example, both the thread structure 15 and the sleeve area 13 can be completely composed of an artificial trabecular structure, thus further improving the cell lifting function.

FIGS. 9 and 10 show a simplified top view and a simplified partial side view of the joint implant according to FIG. 7, wherein the same reference numbers indicate the same or corresponding elements, and a repeated description thereof will therefore be dispensed with below. According to FIGS. 9 and 10, a cross slot in particular can be configured in the cover area 12 of the joint implant 1 as a recess 16 for applying torque. A slot width b of the recess 16 is for example 0.5 mm to 1 mm. The slots can be configured over the entire area of the hollow cylinder wall and optionally the thread structure 15.

Alternatively to the above-described embodiments (slot and cross slot), the recess 16 can also be configured in further forms (not shown). In particular, the recess 16 can have the form of an inner polygon (square, hexagon, etc.) or a Torx. In such an embodiment, the recess 16 may be configured not only locally in the cover area 12, but also as a continuous recess from the base area 11 to the cover area 12 in the hollow space of the rod-shaped body, thus further enlarging the surface of the rod-shaped body and further improving the lifting function for the transport of cells. In order to insert the screw-shaped joint implants 1 in a controlled manner, they can be screwed in for example using pre-inserted Kirschner wires.

Preferably, the joint implants 1 are configured as microstructured rods based on medically approved, bioinert and biocompatible 3D-printable materials such as for example non-bioresorbable polymers, in particular polyamide (PA), polyether ketones, in particular PEK [polyether ketone], PEKK [poly(ether ketone ketone)], PEEK [polyether ether ketone], polyethylene (PE), in particular UHMWPE [ultra high molecular weight polyethylene], or e.g. bioresorbable polymers, in particular PCL [poly-ε-caprolactone].

Alternatively, metals and metal alloys, preferably those suitable for 3D printing, in particular titanium (pure titanium grade 1), in particular Ti64 (Ti6Al4V), Ti64 ELI and TiCP, stainless steel, in particular 316L, and cobalt-chromium alloys, in particular CoCr, can also be used as materials for the joint implants 1 and in particular for their artificial trabecular structures.

Furthermore, non-bioresorbable ceramics, preferably suitable for 3D printing, in particular aluminium oxide [Al₂O₃], and zircon dioxide [ZrO₂] ceramics, or bioresorbable ceramics, in particular calcium phosphate [Ca₃(PO₄)₂] ceramic, can also be used as materials for the joint implants 1.

Preferably, Si₃N₄ can be used as a material for the joint implant 1.

Generally speaking, further medically approved, bioinert and biocompatible materials, in particular suitable for 3D printing, can also be used for the joint implants 1 and in particular for the artificial trabecular structures 14 according to FIGS. 3A to 3F.

FIG. 11 shows a schematic overview of the various differentiation stages of mesenchymal stem cells according to Aubin 1998. According to the invention, it is desirable for mesenchymal stem cells (MSC) to differentiate into chondrocytes at least in the cover area 12 of the rod-shaped body of the joint implant 1 in order to achieve the new cartilage formation desired in this area. On the other hand, differentiation of the mesenchymal stem cells (MSC) into osteocytes can be advantageous in the lower part or base area 11 of the rod-shaped body of the joint implant 1 in order to facilitate bone formation and thus optimum growth of the joint implant 1 into the cancellous bone area 3.

Surprisingly, it was found that such differentiation of mesenchymal stem cells can already be facilitated by producing a corresponding substrate. More specifically, it was found that a hydrophobic surface of a substrate facilitates chondrocyte differentiation of mesenchymal stem cells and thus cartilage formation, while a hydrophilic surface of a substrate or base facilitates osteoblast differentiation of mesenchymal stem cells and thus bone formation.

The terms “hydrophobicity” or “hydrophobic surface” and “hydrophilicity” or “hydrophilic surface” are defined below based on the so-called contact angle of a water droplet on a surface. Here, hydrophobic surfaces show a contact angle of greater than or equal to 90°, wherein contact angles of greater than 160° characterise superhydrophobic surfaces. The most widely-known representative of these superhydrophobic surfaces is the so-called “lotus plant”, which because of its particular micro- and nanostructuring has a contact angle of up to 170°. On the other hand, hydrophilic surfaces are characterized by a contact angle of less than 90°.

According to the invention, this differentiation property of the stem cells as a function of the hydrophobicity or hydrophilicity of a surface is utilized in that the rod-shaped body of the joint implant 1 has corresponding hydrophobic surfaces that facilitate chondrocyte differentiation of the mesenchymal stem cells and thus cartilage formation.

Here, the entire rod-shaped body can have a hydrophobic surface, but it is also possible for only a part of the body to have hydrophobic surfaces. For example, at least the cover area 12 has a hydrophobic surface in order to facilitate cartilage growth at this site. On the other hand, the rod-shaped body can also have a hydrophobic surface in its cover area 12, upper sleeve area 13 and upper thread area 15, while the base area 11, the lower part of the sleeve area 13 and the lower part of the thread area 15 have a hydrophilic surface. In this manner, cartilage growth can be facilitated in the upper area of the joint implant 1 (the area protruding from the bone) and bone growth can be facilitated in the lower area of the joint implant (the area located in the bone).

According to the invention, a surface that is hydrophobic and thus facilitates chondrocyte differentiation can be implemented in a variety of ways. On the one hand, chemical coatings can be applied to the rod-shaped body, and in particular its trabecular structures, which improves hydrophobic (water-repelling) properties. On the other hand, suitable micro- and/or nanostructuring of the surface of the rod-shaped body and in particular its trabecular structures can also produce a desired hydrophobic property. Moreover, combinations of such hydrophobic chemical coatings with physical topography structuring (micro- and/or nanostructuring) can also be used to implement such “lotus effect” surfaces with hydrophobic properties in order to facilitate chondrocyte differentiation and thus cartilage growth.

FIGS. 12A and 12B illustrate the production of such a hydrophobic coating using the example of segmented polyurethanes such as those that can be applied to a joint implant according to the invention.

According to FIG. 12A, the production of NCO-terminated prepolymers is first illustrated, wherein there is a stoichiometric excess of —NCO. According to FIG. 12B, the NCO— terminated prepolymers are then converted using dodecane diol as a non-polar “chain extender” into the desired segmented polyurethane (segmented PU).

FIG. 13A shows an enlarged view of a Ti substrate coated with such a segmented PU. While an uncoated Ti substrate (not shown) has a contact angle of 0°, the Ti surface coated with segmented PU (10% PU in toluene) has contact angle of approx. 112° to 116°.

FIG. 13B shows an enlarged view of a Ti substrate coated with a segmented PU, wherein the concentration of the segmented PU is 2% in toluene. The Ti surface coated with such segmented PU now has a contact angle of approx. 109° to 111°.

The following components were used for the above-described hydrophobic PU coating:

a) aliphatic diisocyanates: isophorone diisocyanate (IPDI), hexamethylene diisocyanate (HDI) and dicyclohexylmethane diisocyanate (hydrogenated MDI, HMDI)

b) polyols: polycarbonate diols (hydrolysis resistance) such as e.g. Desmophen C2200, Desmophen XP2586, and hydrocarbon diols based on natural rubber and hydrogenated natural rubber

c) chain extenders: aliphatic diols such as hexanediol, decanediol and possibly longer diols because of their hydrophobicity

Furthermore, a PU-based polyelectrolyte complex can also be used as a hydrophobic chemical coating. Here, the same components as mentioned above are used, wherein sulfonated diols or ammonium-group-containing diols are additionally used as chain extenders in order to introduce ionic groups for forming the electrolyte complexes.

Complex formation then takes place after coating (preferably dip coating) by immersion in a dilute solution with a surfactant (cationic or anionic, depending on which ionic groups are present in the polymer). The ionic interactions between the polyelectrolytes and the surfactant lead to a solid bond, particularly when the surfactant is hydrophobic and thus already has no tendency to dissolve in an aqueous environment.

Furthermore, an acrylate-based polyelectrolyte complex can also be used as a hydrophobic chemical coating, wherein a first layer of polyelectrolytes such as polyacrylic acid or copolymers containing acrylic or methacrylic acid, optionally also with their own phosphoric acid groups (comonomer vinylphosphonic acid) is applied to the surface for adhesion and a second layer is then applied as described above (a coating of a surfactant solution, coordinated with the ionic groups of the polyelectrolyte).

Preferably, the following three types of hydrophobic coating materials are used:

crosslinked polyurethane,

uncrosslinked polyurethane, and

polyelectrolyte complexes,

which have the following characteristics:

Crosslinked polyurethane:

Polyurethanes differ from most other polymers and plastics in that they are composed of a “modular system” of many different components (diisocyanates, polyisocyanates, polyols, chain extenders, soft segments etc.). The actual construction thereof (chemical synthesis of the polymer molecules) typically occurs only during processing, so that the user or manufacturer of components based on polyurethanes can assemble the final properties in a manner tailored to his requirements. Almost all other plastics, in contrast, are produced and supplied with fixed property profiles by the raw material manufacturer (chemical industry), so that the user or producer of components has only a relatively minimal influence on their property profile. For this reason, polyurethanes constitute a highly favourable starting point for special developments such as the coating of the implant body according to the invention 1.

Polyurethanes have long been used as biocompatible active compounds, for example as inert, non-degradable coatings for cardiac pacemakers, or also as biocompatible, degradable carrier materials (scaffolds) for tissue engineering or regenerative medicine. In such use, the properties thereof (e.g. hydrophobicity/hydrophilicity, degradability/long-term stability, strength, stiffness, porosity etc.) are adjusted by combining the components as required.

Crosslinked polyurethanes are produced in a dilute solution in the presence of the substrate to be coated. In this process, the components can be selected in such a way that chemical bonding to the surface of the substrate to be coated also takes place simultaneously during cross-linking. These materials often show outstanding adhesion without requiring adhesion promoters or similar intermediate layers, particularly to hydrophilic surfaces. The components of the polyurethanes can be selected such that the layers produced are themselves hydrophobic.

Suitable components are aliphatic di- and polyisocyanates for biocompatibility, soft segments and polycarbonate-, silicone- or polybutadiene-based polyols for long-term stability, and long-chain diols, possibly also silicone- or polybutadiene-based, as chain extenders for hydrophobicity.

The main drawback is the problematic control of the layer thickness during coating. The concentration is the only independent parameter that can be varied to influence the layer thickness. Although the composition and the reaction time affect the layer thickness, the composition also affects all of the other properties, and the reaction time cannot be set to any desired duration, because complete reaction of the isocyanate groups is necessary for biocompatibility, so the coating time cannot be reduced to any desired duration.

Uncrosslinked polyurethanes:

Uncrosslinked polyurethanes are produced separately from the coating process and then transferred from a dilute solution to an immersion process. The setting of properties offers the same possibilities as in the case of the crosslinked polyurethanes, as almost all of the components can be used in both cases.

The advantage of the uncrosslinked polyurethanes is that synthesis and coating take place separately from each other, so that there are better possibilities for controlling the layer thickness. The concentration, the exposure time during the immersion process, and above all the number of immersions (with respective drying steps between them) determine the thickness of the applied layer.

The drawback of this is that chemical bonding of the layer of the substrate requires either an adhesion promoter layer or special components in the polyurethane that can react with the surface. In some cases, the adhesion of these layers is thus less long-lasting, or the coating process is more expensive, because an adhesion promoter layer must be added before the actual coating is carried out. However, as this is presumably also possible as a simple immersion coating, the additional expense is limited.

Polyelectrolyte complexes:

Polyelectrolyte complexes utilize electrostatic interactions between positively and negatively charged ions and surfaces. Each material has a specified surface charge in water (its zeta potential), which—depending on the chemical structure—is either positive or negative. Neutral particles or surfaces also possess this surface charge. Polyelectrolytes having charges along the polymer chain that are opposite to this surface charge adhere very strongly to the surface. In general, they can no longer be removed, because each polymer chain, depending on its chain length, adheres simultaneously with dozens or hundreds of groups and is therefore maintained in position even if some of these groups are dissolved due to external influences. As one possibility, oppositely charged polyelectrolytes can then be deposited on these polyelectrolytes, thus making it possible to precisely set the layer thickness on a molecular basis by alternating deposition (layer by layer technology). Alternatively, low-molecular-weight ions, e.g. surfactants or soaps, are deposited, one end of which carries a charge opposite to that of the polyelectrolyte in order to ensure adhesion, and the other end of which is hydrophobic. In the ideal case, layers can be produced in this manner that show a thick layer of e.g. methyl groups toward the outside, thus making it possible to achieve surface tension that is almost identical to that of fluoropolymers (PTFE, Teflon).

The advantages of these materials lie in their ordinarily outstanding adhesion in aqueous or non-aqueous systems, in the precise controllability of the layer thickness, and in their relatively easy-to-control, highly pronounced hydrophobicity.

A drawback is the deposition in almost monomolecular layers, which in the case of larger layer thicknesses requires a large number of immersions in alternating polyelectrolyte baths. However, as drying steps are not required between these immersions, the expense is acceptable.

Furthermore, as a starting material for the rod-shaped body of the joint implant 1, 3D-printable materials can be used that already show a hydrophobic surface per se (e.g. without additional micro- and/or nanostructuring and/or chemical coating). For example, the untreated surface of a zircon dioxide ceramic substrate already has hydrophobic properties.

Alternatively or additionally to the above-described hydrophobic chemical coating or material selection, further micro- and nanostructured surfaces can be produced on the rod-shaped body of the joint implant 1, such as those also known as self-cleaning surfaces under the term Lotus Effect®. Such micro- and nanostructured surfaces are known for example from the document WO 96/04123 A.

Moreover, the artificial trabecular structures 14 can comprise an additional growth coating or a growth factor in order to improve cartilage differentiation and the growth of cartilage material. Preferably, the artificial trabecular structure 14 can be coated with specific human and human homologous growth factors, FGFs [fibroblast growth factors], in particular FGF-1, FGF-2 and FGF-10 to FGF-22 and in particular FGF-18. Alternatively, the artificial trabecular structure 14 can be coated with specific human and human homologous growth factors, SDFs [stromal cell-derived factors], in particular SDF-1. Furthermore, specific human and human homologous growth factor, IGF-1 [insulin-like growth factor 1], human PDGF [platelet-derived growth factor], the specific human and human homologous growth factors TGF-β1 and TGF-β3 [transforming growth factors beta 1 and beta 3] or specific human and human homologous BMP-2 and BMP-7 [bone morphogenetic protein-2and protein-7] can be applied to the artificial trabecular structure 14. Further possibilities for the coating include specific human and human-homologous OP-1 [osteogenic protein-1], human PRP [platelet-rich plasma] and bioinert polyamides especially suitable for coatings. Of course, combinations of the above-described coatings are also possible. Preferably, the growth factor can be applied as the last layer.

According to the invention, by correspondingly selecting suitable bioinert and biocompatible materials with ideal adaptation of the geometric and chemical/biochemical surface structure (artificial trabecular structure), the differentiation of mesenchymal stem cells into chondrocytes or osteoblasts can be selectively controlled. This allows the cartilage structure on the side of the joint implants 1 facing the synovia in particular (cover area 12) to be improved by means of the above-described hydrophobic surface structures and coatings and the growth factors that stimulate cartilage formation. Moreover, on the side of the joint implants facing away from the synovia (base area 11), bone formation and bone structure in the cancellous bone area can be improved by means of hydrophilic surface structures and coatings. In this manner, one achieves almost physiological suitability, as the joint anatomy and natural bone stability are not or only minimally affected such as e.g. in the case of implantation of an endoprosthesis. The compatibility and efficacy of the curative therapy is thus significantly improved by means of the above-described joint implants.

Because of its cylindrical shape with an external thread and its hydrophobic surface, the joint implant according to the invention allows and promotes the capacity of cells from the bone marrow to rise along the rod in order to reach the tissue located around and over the rod. The rod body can be hollow or solid. The joint implant is screwed into the bone and supplies the bone and the cartilage tissue overlying the bone. In particular, the joint implant allows the transport of cells, even if no connecting tissue is present. This allows new tissue esters to be produced, such as e.g. in the case of prolonged arthrosis, or the inflammatory bone, e.g. in the case of oedema, can heal the bone tissue via cellular access.

As the joint implant remains in the placed bone or tissue, the cell transport can occur continuously. This thus allows a quasi-curative treatment.

The joint implant according to the invention allows the transport of cells from the bone tissue to the tissue surface (the so-called lifting function). By means of various structural factors, the surface of the rod can be significantly enlarged, allowing more cells to reach the surface (cover area). The structural factors include for example macrostructuring, such as e.g. channel formation in the interior of the implant body or e.g. the basic form of the thread structure, and micro- to nanostructuring, such as e.g. trabecular microstructuring, but also other types, e.g. channel-like microstructuring on the exterior and optionally the interior of the rod-shaped body. The macro- and micro- to nanostructuring preferably takes place by means of suitable 3D printing processes and optionally subsequent chemical treatment of the surfaces. This considerably enlarges the surface of the rod, allowing more cells to reach the surface. The rod should preferably end flush with the tissue surface of the bone. Alternatively, the rod can end slightly below or slightly above the surface. This allows the cells at the tissue surface/boundary to form new tissue from the bone and bone marrow.

In addition, the joint implant according to the invention can also be used for the fixation of supporting structures (so-called scaffolds) and tissues such as tendons and ligaments to the bone. It can also be used in plastic surgery, wherein, however, as a carrier of cells and a binder of tissues or tissues and medicinal products and materials, it can also be used for the formation of collagenous supporting tissue (callus formation). e.g. in facial surgery.

In addition, the joint implant according to the invention can also bind together separated bone tissue layers, such as e.g. in bone fractures. In this case, by means of suitable macro-, micro- and nanostructuring of the surfaces and/or suitable chemical aftertreatment and/or coating, the hydrophobicity, even on an alternating basis inside the rod, can be set (adjusted) so that optimum colonisation with bone or cartilage cells is achieved in each case.

Moreover, the joint implant does not limit the administration of anti-resorptive agents such as anti-RANKL antibodies (denosumab) or cathepsin K inhibitors or antibodies (odanacatib) or bisphosphonates, in particular bisphosphonates and in particular parenteral bisphosphonates such as ibandronic acid, zoledronic acid and pamidronic acid and bone-strengthening (bone anabolic) drugs. It allows, in addition to the combination with antiresorptive agents and/or bone anabolic agents such as e.g. teriparatide (Forsteo®, PTH 1-84 (Preotact®) or abaloparatide (Tymlos) or anti-sclerostin antibodies (romosozumab, AMG 785)), the combination with e.g. hyaluronic acid, but also other damping fluids or gels as synovial fluid replacements.

The combination of the above-described biocompatible, bioinert, 3D-printable materials, the specifically suitable biomedical geometries (curvature, nanostructure, microstructure and macrostructure) and the growth-promoting coatings provides a novel joint implant that can further optimize the quantity and quality and thus the load-bearing capacity and durability of replacement cartilage tissue and make a substantial contribution to the curative treatment of joint diseases (arthrosis).

The invention was described above by means of preferred examples. However, it is not limited thereto, and in particular also comprises individual combinations of the above-described examples. In particular, carbon can also be used as a material for the joint implants, and in particular for the artificial trabecular structures. In particular, instead of the described cylindrical shape, a slightly conical shape can also be used for the rod-shaped body of the joint implant. In particular, the preliminary stage of chondrocyte differentiation of mesenchymal stem cells, namely chondroblast differentiation of mesenchymal stem cells, can also be facilitated by the hydrophobic surface.

Although the invention has been described above in the context of use in human hip and knee joints, it is not limited to this application and in particular also includes small and extremely small human joints (e.g. foot and finger joints) and animal joints.

### LIST OF REFERENCE NUMBERS

1 Joint implant

2 Articular cartilage

3 Cancellous bone area

4 Periosteum

5 Cortical bone

6 Medullary cavity

7, 8 Trabeculae

11 Floor area

12 Cover area

13 Sleeve area

14 Artificial trabecular structure

15 Thread structure

16 Recess

GD Thread diameter

GS Thread pitch

AD External diameter

ID Internal diameter

L Length of the joint implant

d Thickness of the sleeve area

b Width of the recess

We claim:

1. A joint implant for new tissue formation at a joint, wherein the joint implant (1) comprises a rod-shaped body with a base area (11), a cover area (12) and a sleeve area (13),
wherein
at least the cover area (12), in particular the entire rod-shaped body, of the joint implant (1) has a hydrophobic surface for facilitating chondrocyte differentiation of mesenchymal stem cells; and
a thread structure (15) is at least partially formed on the sleeve area (13) of the joint implant (1).

2. The joint implant according to claim 1, wherein at least the cover area (12) and the thread structure (15) of the joint implant (1), in particular the entire rod-shaped body, have an artificial trabecular structure (14) produced by a 3D printing process.

3. The joint implant according to claim 1, wherein the rod-shaped body of the joint implant (1) is a hollow body with at least one or a plurality of hollow spaces.

4. The joint implant according to claim 1, wherein the rod-shaped body of the joint implant (1) has a cylindrical or conical shape.

5. The joint implant according to claim 1, wherein the material of the rod-shaped body comprises
a polymer, in particular PA, PEK, PEKK, PEEK, UHMWPE or PCL,
a metal, in particular Ti, stainless steel, or a metal alloy, in particular Ti64 or CoCr,
a ceramic, in particular Al₂O₃, ZrO₂ or Ca₃(PO₄)₂,
Si₃N₄, or
combinations of these materials.

6. The joint implant according to claim 1, wherein at least the cover area (12) of the joint implant (1) comprises a recess (16) for applying torque.

7. The joint implant according to claim 5, wherein the recess (16) has the form of a slot, cross slot, inner polygon or Torx.

8. The joint implant according to claim 1, wherein the thread structure (15) of the joint implant (1) has a thread pitch (GS) of 0.5 mm to 1 mm.

9. The joint implant according to claim 1, wherein the base area (11) of the joint implant (1) has a point and the thread structure (15) has a self-forming, self-tapping or self-cutting configuration.

10. The joint implant according to claim 1, wherein the hydrophobic surface is implemented by means of a hydrophobic chemical coating.

11. The joint implant according to claim 10, wherein the hydrophobic chemical coating comprises a segmented polyurethane or polyelectrolyte or a hydrophobically functionalized chitosan or chitosan derivative.

12. The joint implant according to claim 1, wherein the hydrophobic surface is implemented by hydrophobic nanostructuring of a substrate.

13. The joint implant according to claim 1, wherein the rod-shaped body of the joint implant (1) has a length (L) of 0.6 cm to 2.2 cm, in particular 1.25 cm.

14. The joint implant according to claim 1, wherein the rod-shaped body of the joint implant (1) has a thread diameter (GD) of 2 mm to 6 mm, in particular 3 mm.

15. The joint implant according to claim 1, wherein at least the cover area (12), in particular the entire rod-shaped body, of the joint implant (1) comprises a growth factor for facilitating chondrocyte differentiation of mesenchymal stem cells, in particular FGF-1, FGF-2, FGF-10 to FGF-22, SDF-1, IGF-1, PDGF, TGF-β1 and TGF-β3, BMP-2 and BMP-7, OP-1, PRP or bioinert polyamide.

16. The joint implant according to claim 1, wherein at least the base area (11) of the joint implant (1) has a hydrophilic surface for facilitating osteoblast differentiation of mesenchymal stem cells.

17. The joint implant according to claim 1, wherein the material of the rod-shaped body comprises:
a polymer selected from the group consisting of PA, PEK, PEKK, PEEK, UHMWPE, and PCL;
a metal selected from the group consisting of Ti, stainless steel, and a metal alloy;
a ceramic selected from the group consisting of Al₂O₃, ZrO₂ or Ca₃(PO₄)₂, Si₃N₄; or a combinations of these materials.
